(19) United States
(12) Patent Application Publication
(10) Pub. No.: US 2026/0318934 A1
(43) Pub. Date: Oct. 15, 2026

(54) Methods for the Assessment of Lipoprotein Lipase Deficiency and Lipoprotein Lipase Bioavailability

(71) Applicant: 9163-0384 QUÉBEC INC., Chicoutimi (CA)

(72) Inventors: Daniel GAUDET, Chicoutimi (CA); Miriam LAROUCHE, Laterriere (CA); Etienne KHOURY, Chicoutimi (CA); Diane BRISSON, Chicoutimi (CA)

(73) Assignee: 9163-0384 QUÉBEC INC., Chicoutimi (CA)

(21) Appl. No.: 19/116,626

(22) PCT Filed: Oct. 2, 2023
(86) PCT No.: PCT/IB2023/059884

§ 371 Date: Mar. 28, 2025

Related U.S. Application Data
(60) Provisional application No. 63/411,929, filed on Sep. 30, 2022.

(51) Int. Cl. G01N 33/92

(52) U.S. Cl. CPC G01N 33/92; G01N 2333/92; G01N 2800/044

(57) ABSTRACT

Described herein are methods and kits directed to the use of ANGPTL3, ANGPTL4, ANGPTL8 and/or ANGPTL3/8 inhibitors in the field of severe hypertriglyceridemia, particularly to distinguish between complete or partial lipoprotein lipase (LPL) deficiency and assess LPL bioavailability in subjects. As described, these inhibitors can be used to reveal subjects with a significant basal LPL activity from those subjects without any LPL activity or very few LPL activities. The inhibitors can also be used for distinguishing between recurrent chylomicronemia and sustained chylomicronemia in subjects with severe hypertriglyceridemia or chylomicronemia. Administration of the inhibitors can replace post-heparin LPL activity measurement(s) or genotyping in the identification of disorders of triglyceride and HDL-cholesterol metabolism.

### CROSS REFERENCE TO RELATED APPLICATION

The present international PCT application is filed concurrently with U.S. provisional patent application No. 63/587,192 entitled “ASSESSMENT OF CHYLOMICRONEMIA USING VITAMIN E” filed on Oct. 2, 2023, the content of which is incorporated herein by reference in its entirety.

### FIELD OF THE INVENTION

The invention relates to the field of lipoprotein lipase (LPL) deficiency and more particularly to distinguish between complete or partial lipoprotein lipase (LPL) deficiency and assess LPL bioavailability in subjects.

### BACKGROUND OF THE INVENTION

Hypertriglyceridemia is characterized by the accumulation in the blood of lipoproteins rich in triglycerides (TG) originating from the exogenous (chylomicrons and remnants) or endogenous (VLDL and remnants) metabolic pathways. There are several forms of hypertriglyceridemia and the most severe form, hyperchylomicronemia, is defined by the blood accumulation of chylomicrons, the lipoproteins transporting dietary fat after a meal. Chylomicronemia is therefore a postprandial metabolic disturbance characterized by a triglyceridemia greater than 10 mmol/L. The prevalence of chylomicronemia is estimated at 1 person out 600 having this condition (all causes) in the Western world. Chylomicronemia can be sustained or recurrent and several genetic, physiological, environmental factors or health conditions can favor the appearance of this condition. Chylomicronemia is considered to be sustained when the triglyceridemia is most often higher than 10 mmol/L and refractory to conventional hypolipidemic treatments.

Complete lipoprotein lipase (LPL) deficiency is a well-known form of sustained chylomicronemia, and it is the most severe. It is associated with familial hyperchylomicronemia syndrome (FCS), a rare inherited condition that affects approximately 1 to 3 people/1,000,000. Loss-of-function mutations in the LPL gene or genes modulating LPL activity such as APOCII, APOAV, LMF1, GPIHBP1, have previously been identified as causative of the disease, but several mutations in genes involved in the TG metabolic pathways or a high polygenic score can also cause severe hypertriglyceridemia or sustained chylomicronemia. Patients with sustained chylomicronemia (FCS or not) are at very high risk of recurrent acute pancreatitis and other morbidities.

With the increasing event of personalized medicine, it is expected that access to innovative treatments of severe hypertriglyceridemia, chylomicronemia or LPL deficiency will increasingly be restricted to patients having a genotypic confirmation of hyperchylomicronemia, e.g., those subjects carrying two null genetic mutations causing a LPL loss of function or those subjects having an absence of bioavailable LPL protein as confirmed by a post-heparin LPL activity assay. However, available assays for the determination of post-heparin blood LPL activity are invasive or have limitations in one or another of the aspects of reproducibility, validation, antibody sensitivity, substrate stability or environmental friendliness.

Another problem with LPL is that genotypic confirmation is not always consistent with phenotypic disease expression. Indeed, several factors can disrupt the activity of LPL and cause sustained chylomicronemia or severe hypertriglyceridemia: an additional unidentified and/or undocumented mutation in the LPL gene and/or in a gene of the same biological pathway; epigenetic modifications preventing adequate transcription of the LPL gene(s); circulating antibodies against LPL, secondary factors, combinations of primary and secondary factors, etc. Therefore, patients with sustained chylomicronemia or severe hypertriglyceridemia who do not carry null FCS causing mutations may therefore not have a proper access to appropriate treatments, despite a phenotypic and clinical profile identical to that of FCS patients.

Angiopoietin-like proteins (ANGPTLs) are a family of proteins. ANGPTL3 is one of the most documented ANGPTL and it is an important regulator of lipid metabolism because this protein inhibits the action of LPL. In the last decade, ANGPTL3 has emerged as a novel therapeutic target for lowering blood LDL-C and triglycerides. Many ANGPTL3 inhibitors such as monoclonal antibodies, antisense oligonucleotides and small-interfering ribonucleic acids (siRNAs) are currently available or in clinical trials and/or have shown great promises for treating a wide range of lipid diseases. To be effective ANGPTL3 inhibitors critically require the presence of LPL since they are LPL-dependent (Ahmad Z, et al. J Am Coll Cardiol. 2021 Jul. 13; 78(2): 193-195). After a meal, ANGPTL3 interacts with ANGPTL8, and this tandem has a huge effect on LPL. Because of the LPL-dependency, the efficacy of dual ANGPTL3 and ANGPTL 8 inhibition also depends on the presence of LPL. This tandem increases by up to 100-fold the lipolysis through LPL pathway.

Angiopoietin-like protein-4 (ANGPTL4) is also known to inhibit LPL. However, unlike ANGPTL3 and ANGPTL8 which are effective in a fed state, ANGPTL4 may have an important role in fasted state. Its paracrine role is almost exclusive to adipose tissue and phase I clinical trials began in June 2022.

Although the new ANGPTLs inhibitors therapies seem very promising for the reduction of LDL-cholesterol, remnants and/or triglycerides, the use and development of ANGPTL3 inhibitors, ANGPTL4 inhibitors or ANGPTL3 and/or ANGPTL8 inhibitors are solely therapeutic. The use of such inhibitors for diagnostic purposes or for assessing LPL bioavailability, or to clinically and qualitatively replace post-heparin LPL measurement, or to distinguish between complete or partial LPL deficiency or the like has never been suggested.

Several existing or emerging treatments for severe hypertriglyceridemia such as ANGPTL3, ANGPTL4 or ANGPTL3/8 inhibitors require the presence of LPL to be effective, whereas others are LPL-independent (e.g., APOC3 inhibitors). Given the chronicity of the susceptibility to hypertriglyceridemia, the half-life and the cost of the treatments, assessing the bioavailability of LPL is therefore essential to establish the odds of response and assess an expected TG-lowering effect of therapeutics agents.

For instance, AAV-based LPL gene replacement therapy for patients with LPL deficiency and sustained chylomicronemia is administered once in a lifetime solely in patients with complete LPL deficiency. The right of access to treatment, requires the demonstration of the absence of LPL bioavailability, not only the genetic background. Conversely, genome editing strategies (e.g., CRISPR-Cas or others) targeting ANGPTL3 inhibition are likely to be administered every 10 years or once in a lifetime and critically require the presence of LPL bioavailability to establish the right of access to the treatment.

There is thus a need for new methods and assays to assess lipoprotein lipase (LPL) deficiencies or assess LPL bioavailability, that are not invasive, sensitive, stable and/or user-friendly. There is also a need for methods and assays which could replace, from a clinical standpoint, pots-heparin LPL techniques or other techniques that are not validated and lack reproducibility.

There is thus a need for new methods and assays allowing to give access to personalized medicine treatments of hypertriglyceridemia, chylomicronemia or LPL deficiency not only to patients having a genotypic confirmation of the diseases.

There is particularly a need for methods allowing to distinguish between complete or partial lipoprotein lipase (LPL) deficiency in subjects, for instance patients with a complete LPL deficiency vs. patients having a residual activity of this protein.

There is also a need for diagnosing multifactorial chylomicronemia and/or familial hyperchylomicronemia syndrome (FCS) in subjects.

There is also a need for assessing LPL bioavailability in patients with sustained chylomicronemia and/or familial hyperchylomicronemia syndrome (FCS).

There is also a need for improved methods for treating a subject suffering from complete or partial lipoprotein lipase (LPL) deficiency or lacking bioavailable LPL.

The present invention addresses these needs and other needs as it will be apparent from the review of the disclosure and description of the features of the invention hereinafter.

### BRIEF SUMMARY OF THE INVENTION

According to one particular aspect, the invention relates to a method to distinguish between complete or partial lipoprotein lipase (LPL) deficiency in a subject, comprising: (i) administering to the subject at least one inhibitor of ANGPTL3, ANGPTL4, ANGPTL8, and/or ANGPTL3/8; and (ii) measuring the subject's response to the inhibitor; wherein said response identifies the subject as suffering from a complete or partial deficiency in LPL.

According to another particular aspect, the invention relates to a method for assessing lipoprotein lipase (LPL) bioavailability in patients with sustained chylomicronemia and/or familial hyperchylomicronemia syndrome (FCS), comprising: (I) administering to the patient at least one inhibitor of ANGPTL3, ANGPTL4, ANGPTL8, and/or ANGPTL3/8, and (ii) measuring the patient's response to the inhibitor; wherein a decrease in the patient's blood triglycerides confirms LPL bioavailability, and wherein an absence of decrease of the patient's blood triglycerides is indicative of an absence or rarity of bioavailable LPL.

According to another particular aspect, the invention relates to a method for distinguishing between recurrent chylomicronemia and sustained chylomicronemia in a subject with severe hypertriglyceridemia or chylomicronemia, the method comprising the steps of (i) administering to the subject at least one inhibitor of ANGPTL3, ANGPTL4, ANGPTL8, and/or ANGPTL3/8; and (ii) measuring the subject's response to the inhibitor.

According to another particular aspect, the invention relates to a method for replacing post-heparin LPL activity measurement(s) in the identification of disorders of triglyceride and HDL-cholesterol metabolism, the method comprising substituting said post-heparin LPL activity measurement with administration to the subject of at least one inhibitor of ANGPTL3, ANGPTL4, ANGPTL8, and/or ANGPTL3/8.

According to another particular aspect, the invention relates to a kit to distinguish between complete or partial lipoprotein lipase (LPL) deficiency in a subject.

According to another particular aspect, the invention relates to the use of at least one inhibitor of ANGPTL3, ANGPTL4, ANGPTL8, and/or ANGPTL3/8 for at least one of: (i) distinguishing between complete or partial lipoprotein lipase (LPL) deficiency in a subject; (ii) identifying a subject as suffering from recurrent chylomicronemia, (iii) identifying a subject as suffering from sustained chylomicronemia and (iv) identifying a subject as suffering from hyperchylomicronemia syndrome (FCS).

According to additional particular aspect, the invention relates to the uses of at least one inhibitor of ANGPTL3, ANGPTL4, ANGPTL8, and/or ANGPTL3/8 for: (i) identifying in a human subject a genetic mutation causing a complete LPL deficiency; (ii) for identifying human subjects susceptible to respond to LPL-dependent agents; (iii) for identifying human subjects likely not responding to LPL-dependent agents; (iv) for identifying human subjects qualifying for ANGPTL3 inhibition genome editing strategies; (v) for identifying human subjects qualifying for LPL gene replacement therapy or LPL genome editing; and/or for (vi) for substituting a post-heparin LPL activity measurement test in the identification of disorders of triglyceride and HDL-cholesterol metabolism.

According to additional particular aspect, the invention relates a method for treating a subject suffering from complete or partial lipoprotein lipase (LPL) deficiency. In one embodiment the method comprises: (i) administering to the subject at least one inhibitor of ANGPTL3, ANGPTL4, ANGPTL8, and/or ANGPTL3/8; (ii) evaluating the subject's response to the inhibitor by measuring the subject's triglyceridemia or the subject's level of chylomicrons in blood, wherein said response identifies the subject as having or not bioavailable LPL; and (ill) selecting a treatment plan taking into consideration said response.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE FIGURES

For the invention to be readily understood, embodiments of the invention are illustrated by way of example in the following FIGURE.

FIG. 1 is a scattered graph showing a linear relation between FCS diagnosis scores by using scoring system model A (circles) and model B (triangles) and post-heparin plasma LPL activity*. LPL, Lipoprotein lipase; FCS and arrows represent, Familial chylomicronemia syndrome diagnosis scores cut-off in Model A (left side) and model B (right side). *Mean was used for subjects for which LPL activity was obtained using both methods.

Further details of the invention and its advantages will be apparent from the detailed description included below.

### DETAILED DESCRIPTION OF EMBODIMENTS

In the following description of the embodiments, references to the accompanying FIGURES are illustrations of an example by which the invention may be practised. It will be understood that other embodiments may be made without departing from the scope of the invention disclosed. Unless defined otherwise, all technical and scientific terms used herein have the same meaning as commonly understood by one of ordinary skilled in the art to which the invention belongs.

The present invention relates to the field of lipoprotein lipase (LPL) deficiency, and it scientifically relies on the fact that ANGPTL3, ANGPTL4, ANGPTL8 and/or ANGPTL3/8 inhibitors can upregulate lipoprotein lipase (LPL). Therefore, in accordance with the present invention, these inhibitors can be used to reveal subjects with a significant basal LPL activity from those subjects without any LPL activity or very few LPL activities.

In one particular aspect, the invention relates to methods allowing to distinguish between complete or partial lipoprotein lipase (LPL) deficiency. In another aspect, the invention relates to methods for assessing lipoprotein lipase (LPL) bioavailability in patients with sustained chylomicronemia and/or familial hyperchylomicronemia syndrome (FCS).

One aspect of the invention concerns a method to distinguish between complete or partial lipoprotein lipase (LPL) deficiency in a subject. In one embodiment the method comprises: (i) administering to the subject at least one inhibitor of ANGPTL3, ANGPTL4, ANGPTL8, and/or ANGPTL3/8; and (ii) measuring the subject's response to the inhibitor. In accordance with this method, the subject's response identifies the subject as suffering from a complete LPL deficiency or partial deficiency in LPL, i.e., the subject has some bioavailable LPL that can be upregulated by the inhibitor.

Another aspect of the invention concerns a method for diagnosing partial LPL deficiency, multifactorial chylomicronemia and/or complete LPL deficiency in a subject. In one embodiment the method comprises: (i) administering to the subject at least one inhibitor of ANGPTL3, ANGPTL4, ANGPTL8, and/or ANGPTL3/8; and (ii) measuring the subject response to the inhibitor. In accordance with this method, the subject's response identifies the subject as suffering from either partial LPL deficiency, multifactorial chylomicronemia or complete LPL deficiency.

Another aspect of the invention concerns a method for assessing lipoprotein lipase (LPL) bioavailability in patients with sustained chylomicronemia and/or familial hyperchylomicronemia syndrome (FCS). In one embodiment the method comprises: (i) administering to the patient at least one inhibitor of ANGPTL3, ANGPTL4, ANGPTL8, and/or ANGPTL3/8, and (ii) measuring the patient's response to the inhibitor. According to this method a decrease in the patient's blood triglycerides confirms LPL bioavailability, whereas an absence of decrease of the patient's blood triglycerides is indicative of an absence or rarity of bioavailable LPL.

Another aspect of the invention concerns a method for distinguishing between recurrent chylomicronemia and/or sustained chylomicronemia in a subject with severe hypertriglyceridemia or chylomicronemia. In one embodiment, the method comprises administering to the subject at least one inhibitor of ANGPTL3, ANGPTL4, ANGPTL8, and/or ANGPTL3/8; and (ii) measuring the subject's response to the inhibitor. In accordance with this method, the measured response identifies the subject as suffering from recurrent chylomicronemia, sustained chylomicronemia or complete LPL deficiency familial or chylomicronemia syndrome (FCS). Preferably, for more robust conclusions, steps (i) and (ii) are repeated at different intervals (e.g., at least 2 times, at least 3 times or more, the repetition occurring after week, a few weeks, a month, a few months or longer.

Another aspect of the invention concerns a method for replacing post-heparin LPL activity measurement(s) in the identification of disorders of triglyceride and HDL-cholesterol metabolism, the method comprising clinically substituting said post-heparin LPL activity measurement with administration to the subject of at least one inhibitor of ANGPTL3, ANGPTL4, ANGPTL8, and/or ANGPTL3/8.

As used herein, the term “subject” includes a human, more preferably a human patient in need of treatment. Even more preferably the subject is a human patient diagnosed or susceptible to suffer from severe hypertriglyceridemia, including but not limited to sustained chylomicronemia.

The present invention refers to measurement of chylomicrons, vitamin E, and/or triglycerides, etc. in the blood of a subject. In the context of the present invention, the term “blood” broadly encompasses whole blood as well as blood components such as plasma and serum. It is within the skills of those in the art to determine whether measurements must be carried out in whole blood, serum and/or plasma.

As used herein, the term “ANGPTL3” or “Angiopoietin-like 3” refer to a protein that is encoded in humans by the ANGPTL3 gene (Gene ID: 27329). It is also known as ANL3, ANG-5, FHBL2, and ANGPT5.

As used herein, the term “ANGPTL4” or “Angiopoietin-like 4” refer to a protein that is encoded in humans by the ANGPTL4 gene (Gene ID: 51129). It is also known as NL2, ARP4, FIAF, HARP, PGAR, HFARP, TGQTL, UNQ171 and pp 1158.

As used herein, the term “ANGPTL8” or “Angiopoietin-like 8” refers to a protein that is encoded in humans by the ANGPTL8 gene (Gene ID: 55908). It is also known as RIFL, TD26, PRO1185, PVPA599, and C19orf80.

As used herein, the term “ANGPTL3/8” refers to a complex formed by ANGPTL8 with ANGPTL3.

The present invention encompasses the use of any suitable ANGPTL3, ANGPTL4, ANGPTL8, and/or ANGPTL3/8 inhibitor(s) that can upregulate LPL and, consequently, reveal subjects with a basal biological capacity for the LPL from others who have no or very few LPL activities. The inhibitor may be effective against one or more of the ANGPTL3, ANGPTL4, ANGPTL8 proteins or ANGPTL3/8 complex. Suitable inhibitors may include, but are not limited to, antibodies (e.g., monoclonal antibodies), immunogens, gene silencers such as antisense oligonucleotides, small-interfering ribonucleic acids (siRNAs) and microRNAs, small chemical molecules (e.g., oral medicaments), etc. Examples of ANGPTL3 antibodies include, but are not limited to, Evinacumab (Evkeeza™) and ANGPTL3 antibodies described in US 2020/0061189, US 2022/0127348 or US 2022/0153825 (Regeneron Pharmaceuticals). Examples of anti-ANGPTL8 antibodies include, but are not limited to, those described in WO 2017/177181. Examples of anti-ANGPTL3/8 complex antibodies include, but are not limited to, those described in US 2022/0110537. Examples of antisense oligonucleotides that inhibit ANGPTL3 protein synthesis include, but are not limited to, Vupanorsen (Ionis Pharmaceuticals) and those described in WO 2020/099525 (Lipigon Pharmaceuticals AB). Examples of siRNAs directed against ANGPTL3 messenger ribonucleic acid include, but are not limited to, ARO-ANG3 (Arrowhead Pharmaceuticals). Additional examples include microRNA inhibitors of ANGPTL3 and/or ANGPTL8 such as those described in U.S. Pat. Nos. 11,058,710 and 10,844,383 (Dasman Diabetes Institute). It may also be envisioned to use CRISPR-based gene editing technologies (e.g., liposomal or adenoviral-me based editing of ANGPTL3, ANGPTL4 and/or ANGPTL8) or any other technology inhibiting ANGPTL3, ANGPTL4, ANGPTL8 and/or ANGPTL3/8. Preferably, the ANGPTL8 inhibitor(s) is not used alone and is used in combination with inhibitor(s) of ANGPTL3.

In one embodiment, LPL deficiency (e.g., (complete LPL deficiency) is assessed by administering to the subject an ANGPTL3 inhibitor, an ANGPTL4 inhibitor, an ANGPTL8 inhibitor, and/or a ANGPTL3/8 inhibitor for a short-term period (e.g., less than 3 months, or less than 2 months, or less than 1 month).

Any suitable method or route may be used to administer an inhibitor of ANGPTL3, ANGPTL4, ANGPTL8, and/or ANGPTL3/8 to a subject. These routes may include oral, sublingual, rectal, topical, parenteral (e.g., intravenous, intramuscular, subcutaneous), etc. Those skilled in the art can readily identify the best route depending on various factors such as the recommended or medically approved route for administering the inhibitor, potential side effects, rapidity of the desired response, etc.

Many suitable methods, techniques or assays can be used to measure a subject's response to the inhibitor(s). In embodiments, the response to the inhibitor is measured by assessing the subject's triglyceridemia, i.e., levels of triglycerides in the subject's blood, or subject's glycerolemia (i.e., levels of glycerol in the subject's blood). In addition, as described in U.S. provisional patent application No. 63/587,192 entitled “ASSESSMENT OF CHYLOMICRONEMIA USING VITAMIN E” filed Oct. 2, 2023 and incorporated herein by reference, vitamin E is absorbed and transported by chylomicrons. As such, in accordance with the present invention, vitamin E may be used as a valuable surrogate marker of the level of chylomicrons and chylomicron remnants. In embodiments of the methods of the invention described therein, the response to the inhibitor(s) is measured by assessing the subject's blood vitamin E level (i.e., levels of vitamin E in the subject's blood). In embodiments, the subject's response to the inhibitor(s) is measured both, before and after administration of the inhibitor(s). Measurement of triglycerides, free glycerol and/or vitamin E in the blood can be achieved using any suitable medical test.

In one embodiment, assessing lipoprotein lipase (LPL) bioavailability comprises administering the ANGPTL3, ANGPTL4, ANGPTL8, and/or ANGPTL3/8 inhibitor(s) for a short-term period (e.g., less than 3 months, or less than 2 months, or less than 1 month) and measuring the subject's triglyceridemia (e.g., triglycerides in the blood) before and after administration of the inhibitor(s).

In embodiments, the measured response identifies the subject as suffering from a complete or partial deficiency in LPL. In embodiments, the measured response indicates whether LPL is bioavailable or not. The measured response further provides information about the subject's health risk to LPL.

In embodiments, the response to the inhibitor is measured by assessing the subject's triglyceridemia. In embodiments, a lower reduction in blood triglycerides following administration of the inhibitor to the subject is indicative of lower LPL activity. In one particular embodiment, a reduction of at least 20% (e.g., ≥20%, ≥25%, ≥30%, ≥35%, ≥40%, ≥45% or more of blood triglycerides, following administration of the inhibitor(s) is indicative of the presence of LPL activity and LPL bioavailability, therefore discarding a complete LPL deficiency. In embodiments, a reduction of less than 20% (e.g., <20%, <15%, <10%, <5% or less) of blood triglycerides is suggestive of a lack of LPL bioavailability and/or is suggestive of a sustained chylomicronemia. In embodiments, an absence of response or a reduction of less than 5% of blood triglycerides suggests a complete LPL deficiency.

For more certainty, if the reduction in blood triglycerides is about 20% and/or less than 20% it may be preferable to validate the diagnosis, for instance to confirm the status of sustained chylomicronemia or FCS. In embodiments, the methods further comprise using a LPL deficiency diagnosis scoring system for supporting an adequate diagnosis of a complete or partial deficiency in LPL. In embodiments, the method further comprises using vitamin E levels and/or markers of lipolysis and/or assessing circulating lipoproteins in conjunction with the diagnosis scoring system.

Those skilled in the art can easily determine the amount and/or nature of the inhibitor to be administered to the subject. These may depend on various parameters such as age, the patient health condition, fasting conditions, the nature of the inhibitor, the use of a single or multiple inhibitors, etc. For instance, without wishing being bound by theory, it is expected that administering two inhibitors (e.g., an inhibitor of ANGPTL3 plus an inhibitor ANGPTL8) or administering a compound capable of inhibiting both ANGPTL3 and ANGPTL8 will provide a more pronounced effect on LPL upregulation after a meal (post-prandially), and could be more sensitive as a diagnosis test or a test assessing LPL bioavailability than using a single ANGPTL3 inhibitor, since chylomicronemia is a post-prandial phenotype. However, using a single inhibitor (e.g., using and ANGPTL3 inhibitor alone) should be sufficient in most of the cases. It is within the skills of those in the art to select proper dosages, dosage regimens, mode of administration, etc. In embodiments those parameters are in accordance with the approved monography for the therapeutic uses of the inhibitors.

Advantageously, knowing the subject's conditions (e.g., suffering from a complete or partial deficiency in LPL) will allow medical personnel to make better informed decisions regarding a treatment plan to the patient. Accordingly, in embodiments, the method further comprises selecting a treatment plan taking into consideration the subject's response to the inhibitor(s). In embodiments, the treatment plan aims to lower the patient's triglyceridemia and/or chylomicronemia. For instance, if the patient is diagnosed with a complete LPL deficiency or a familial chylomicronemia syndrome (FCS), the treatment plan may comprise using LPL-independent agents such as ApoC3 inhibitors, using LPL gene replacement therapy and or using genome editing strategies, preferably in conjunction with an adapted very low-fat diet+/−medium chain triglycerides (MCT). On the other hand, depending on the clinical phenotype and comorbidities, if the patient is diagnosed with a partial LPL deficiency or multifactorial chylomicronemia, the treatment plan may comprise using either LPL-dependent agents such as ANGPTL3 inhibitors, and/or ANGPTL3/8 inhibitors or using LPL-independent agents, alone or in combination with an adapted diet.

Another aspect of the invention concerns a kit to distinguish between complete or partial lipoprotein lipase (LPL) deficiency in a subject. In one embodiment the kit comprises: (I) an inhibitor of ANGPTL3, ANGPTL4, ANGPTL8, and/or ANGPTL3/8 and (ii) component(s) for measuring the subject's triglycerides and/or for measuring vitamin E and/or for measuring free glycerol blood levels.

As can be appreciated, the present invention provides numerous advantages and benefits. For instance, it makes it possible to diagnose people with multifactorial chylomicronemia, or with other causes of sustained chylomicronemia, or diagnose people having a residual LPL bioavailability, from those individuals with complete LPL deficiency or familial hyperchylomicronemia syndrome (FCS). Advantageously, this is feasible without having to resort to a typical post-heparin LPL activity measurement test which is invasive and known to provide variable results between laboratories. The invention thus provides a non-invasive and reliable alternative to the common post-heparin LPL activity measurement test. Therefore, using an inhibitor of ANGPTL3, ANGPTL4, ANGPTL8, and/or ANGPTL3/8 may be useful for substituting post-heparin LPL activity measurement tests in the identification of disorders of triglyceride and HDL-cholesterol metabolism.

Using an inhibitor of ANGPTL3, ANGPTL4, ANGPTL8, and/or ANGPTL3/8 may also be useful to replace, confirm and/or invalidate a presumptive genetic diagnosis of sustained chylomicronemia or FCS in people requiring such diagnosis. Since all the mutations and factors causing a complete LPL deficiency have not yet been identified nor documented, the present invention may allow the identification of several unconfirmed cases of sustained chylomicronemia, the identification of complete LPL deficiency and/or the identification of FCS. The invention may thus be used for confirming that new genetic mutations are causing or not a complete LPL deficiency. Likewise, the present invention may also be used to demonstrate that a newly identified mutation is not null. For instance, if a new mutation is found, but the person still responds to an ANGPTL inhibitor, it would mean that the mutation is non-null and can increase the severity of the disease rather than causing the disease.

Also, in accordance with the present invention, treatment of patients will be facilitated based on LPL bioavailability.

Those skilled in the art will recognize, or be able to ascertain, using no more than routine experimentation, numerous equivalents to the specific procedures, embodiments, claims, and examples described herein. Such equivalents are considered to be within the scope of this invention and covered by the claims appended hereto. The invention is further illustrated by the following examples, which should not be construed as further or specifically limiting.

### EXAMPLES

### Example 1: Correlation Between Chylomicronemia Diagnosis Scores and Post-Heparin Lipoprotein Lipase Activity

A study was carried out to demonstrate that there is a correlation between FCS diagnosis scoring systems and post-heparin activity.

Briefly, post-heparin plasma LPL activity was measured using colorimetric assays in a sample of 29 subjects with sustained chylomicronemia (20 FCS and 9 MCS). Correlation analyses were conducted to estimate the linear relationship between LPL activity and two diagnosis scoring systems integrating (model A) or not (model B) apolipoprotein B and free glycerol, a surrogate marker of triglyceride hydrolysis.

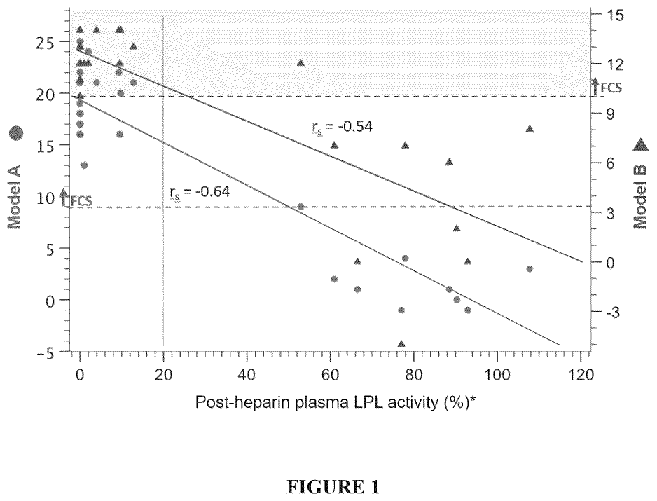

As shown in FIG. 1, there was a significant (p<0.001) difference in post-heparin LPL activity between FCS and MCS. Both scoring systems significantly correlated with post-heparin LPL activity (model A: rₛ=−0.64, p 0.001; model B: rₛ=−0.54, p=0.002).

Overall, when combining post-heparin LPL activity levels issued from both assays, the association between LPL activity and the model A score seems a little stronger than with the model B score (rs=−0.64; p<0.001 vs. rs=−0.54; p=0.002), although the difference between them was not significant (FIG. 1). Sensitivity and specificity in predicting post-heparin LPL activity <20% were the same with both scoring systems (Table 4).

TABLE 4
Performance of model A and model B FCS scoring systems in predicting
post-heparin LPL activity < 20% (95% Confidence interval) *

| | |
|---|---|
| Sensitivity | 100 (83.2-100) |
| Specificity | 88.9 (51.8-99.7) |
| Positive predictive value | 95.2 (76.2-99.9) |
| Negative predictive value | 100 (63.1-100) |
| Area under the ROC curve | 0.94 (0.84-1) |

* Same values were obtained with both scoring systems
FCS, familial chylomicronemia syndrome;
LPL, lipoprotein lipase;
ROC, receiver operating characteristic curve

These results suggest that chylomicronemia diagnosis scoring systems correlate with LPL activity and adequately contribute to distinguish FCS from MCS.

Accordingly, this study confirms that inhibitor(s) of ANGPTL3, ANGPTL4, ANGPTL8, and/or ANGPTL3/8 may be used for substituting a post-heparin LPL activity measurement test in the identification of disorders of triglyceride and HDL-cholesterol metabolism. For instance, when the response to ANGPTL3, 3/8 or 4 inhibitors on the decrease of the blood TG levels is less than 20%, the use of FCS diagnostic scoring systems may confirm the absence or presence of bioavailable LPL.

Therefore, the present invention provides a non-invasive and reliable alternative to the common post-heparin LPL activity measurement test. As such, the invention could clinically replace post-heparin LPL activity measurement for decision-making purposes.

### Example 2: Identification of a New, Unidentified Variant in the LPL Gene Causing FCS

Evinacumab is an angiopoietin like protein 3 (ANGPTL3) monoclonal antibody developed to treat a large spectrum of lipid disorders. As mentioned herein, efficacy of Evinacumab in decreasing blood TG levels depends on LPL bioavailability.

The LPL genotype and drug response in patients with sustained chylomicronemia treated with Evinacumab were assessed.

Briefly, a phase II clinical trial was conducted with Evinacumab to evaluate its safety and efficacy over 20 weeks in patients with severe hypertriglyceridemia and chylomicronemia. There were 3 cohorts in this study, one of which included patients with sustained chylomicronemia and LPL deficiency (LPLD). LPL genotypes were assessed in all patients. Plasma TG values were measured at baseline and every 2 weeks for 20 weeks. Genotype-specific response to Evinacumab was assessed by comparing pre-post TG values.

This phase II trial identified one patient being homozygote for an undocumented mutation (E282X) in the LPL gene who did not respond to Evinacumab (absence of TG decrease after 20 weeks), which is compatible with lack of LPL bioavailability. This variant is thus suspected to cause a LPLD (OMIM: 609708).

The present study thus confirms that, as described herein, inhibitor(s) of ANGPTL3, ANGPTL4, ANGPTL8, and/or ANGPTL3/8 may be used for identifying genetic mutations causing a complete LPL deficiency.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “an inhibitor” includes one or more of such inhibitors and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. A method to distinguish between complete or partial lipoprotein lipase (LPL) deficiency in a subject, comprising: (i) administering to the subject at least one inhibitor of ANGPTL3, ANGPTL4, ANGPTL8, and/or ANGPTL3/8; and (ii) measuring the subject's response to the inhibitor; wherein said response identifies the subject as suffering from a complete or partial deficiency in LPL.

2. The method of claim 1, wherein measuring the subject's response comprises measuring at least one of the subject's triglyceridemia, the subject's glycerolemia and the subject's level of chylomicrons in blood.

3. (canceled)

4. (canceled)

5. (canceled)

6. The method of claim 1,
wherein a reduction of at least 20% of blood triglycerides following administration of the at least one inhibitor is indicative of presence of bioavailable LPL or is indicative of a partial LPL deficiency;
wherein a reduction of less than 20% of blood triglycerides following administration of the at least one inhibitor is indicative of a lack of LPL bioavailability; and.

7. (canceled)

8. (canceled)

9. The method of claim 1, further comprising evaluating the subject with a LPL deficiency clinical diagnosis scoring system for supporting an adequate diagnosis of complete or partial deficiency in LPL.

10. (canceled)

11. (canceled)

12. The method of claim 1, wherein said LPL deficiency is indicative of at least one of multifactorial chylomicronemia, sustained chylomicronemia, complete LPL deficiency and familial chylomicronemia syndrome (FCS).

13. (canceled)

14. (canceled)

15. The method of claim 1, further comprising selected a treatment plan, and wherein the treatment plan comprises lowering the patient's triglyceridemia and/or the patient's glycerolemia.

16. The method of claim 1, wherein said response identifies the subject as suffering from a complete LPL deficiency, wherein the method further comprises selecting a treatment plan, and wherein the treatment plan comprises using LPL-independent agents, such as an ApoC3 inhibitor.

17. The method of claim 13, wherein said response identifies the subject as suffering from a partial LPL deficiency, wherein the method further comprises selecting a treatment plan, and wherein the treatment plan comprises administering to the subject an ANGPTL3 inhibitor and/or an ANGPTL3/8 inhibitor.

18. The method of claim 1, wherein the ANGPTL3, ANGPTL4, ANGPTL8, and/or ANGPTL3/8 inhibitor is selected from the group consisting of antibodies, immunogens, antisense oligonucleotides, small-interfering ribonucleic acids (siRNAs), microRNAs and small chemical molecules inhibiting expression and/or biological activity of at least one of ANGPTL3, ANGPTL4, ANGPTL8 and ANGPTL3/8.

19. (canceled)

20. (canceled)

21. (canceled)

22. A method for assessing lipoprotein lipase (LPL) bioavailability in patients with sustained chylomicronemia and/or familial hyperchylomicronemia syndrome (FCS), comprising: (i) administering to the patient at least one inhibitor of ANGPTL3, ANGPTL4, ANGPTL8, and/or ANGPTL3/8, and (ii) measuring the patient's response to the inhibitor; wherein a decrease in the patient's blood triglycerides confirms LPL bioavailability, and wherein an absence of decrease of the patient's blood triglycerides is indicative of an absence or rarity of bioavailable LPL.

23. The method of claim 22, wherein measuring the subject's response comprises measuring the subject's triglyceridemia.

24. The method of claim 22, wherein measuring the subject's response comprises measuring the subject's glycerolemia.

25. The method of claim 22, wherein measuring the subject's response comprises measuring the subject's level of chylomicrons in blood.

26. The method of claim 25, wherein the subject's level of chylomicrons is measured by assessing levels of vitamin E in the blood.

27. The method of claim 22, further comprising selecting a treatment plan taking into consideration said LPL bioavailability.

28. The method of claim 27, wherein LPL is absent or rare, and wherein the treatment plan comprises using LPL-independent agents such as ApoC3 inhibitors.

29. The method of claim 27, wherein LPL is bioavailable and wherein the treatment plan comprises using LPL-dependent agents such as ANGPTL3 inhibitors.

30. The method of claim 22, wherein the ANGPTL3, ANGPTL4, ANGPTL8, and/or ANGPTL3/8 inhibitor is selected from the group consisting of antibodies, immunogens, antisense oligonucleotides, small-interfering ribonucleic acids (siRNAs), microRNAs and small chemical molecules inhibiting expression and/or biological activity of at least one of ANGPTL3, ANGPTL4, ANGPTL8 and ANGPTL3/8.

31. The method of claim 30, wherein said ANGPTL3 antibody is Evinacumab, wherein said antisense oligonucleotide is Vupanorsen, and wherein said siRNA is ARO-ANG3.

32.-38. (canceled)

39. A kit to distinguish between complete or partial lipoprotein lipase (LPL) deficiency in a subject, comprising: (i) an inhibitor of ANGPTL3, ANGPTL4, ANGPTL8, and/or ANGPTL3/8 and (ii) component(s) for measuring the subject's triglycerides and/or glycerol blood levels.

40.-52. (canceled)
